(19) United States
(12) Patent Application Publication
(10) Pub. No.: US 2005/0033338 A1
(43) Pub. Date: Feb. 10, 2005

(54) Surgical instruments particularly suited to severing ligaments and fibrous tissues

(76) Inventor: Bret A. Ferree, Cincinnati, OH (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 10/873,021

(22) Filed: Jun. 21, 2004

Related U.S. Application Data
(60) Provisional application No. 60/479,718, filed on Jun. 19, 2003.

(51) Int. Cl. A61B 17/32

(52) U.S. Cl. 606/167

(57) ABSTRACT

Surgical instruments particularly suited to severing ligaments, fibrous tissues, and spinal structures feature a shaft terminating in a distal end including a blunt tip for separating tissues to be protected from tissue to be cut, and a sharp cutting blade spaced apart from the blunt tip for severing the tissue to be cut. In alternative embodiments, the distal end may include two blunt tips, one on either side of the sharp cutting blade. In other embodiments, the distal includes an angled portion, and the cutting blade adjoins the angled portion. In further embodiments, the sharp cutting blade may be retractable or covered by a moveable sheath. In all embodiments, the shaft is curved to facilitate easier viewing of the distal end. Novel retractors, other instruments, and endplates for artificial disc replacements are also disclosed.

### REFERENCE TO RELATED APPLICATION

This application claims priority from U.S. Provisional Patent Application Ser. No. 60/479,718, filed Jun. 19, 2003, the entire content of which is incorporated herein by reference.

### FIELD OF THE INVENTION

This invention relates generally to surgery and, in particular, to apparatus for severing posterior longitudinal ligament (PLL).

### BACKGROUND OF THE INVENTION

Spinal surgeons often cut tissues, such as the Posterior Longitudinal Ligament (PLL) that lie over delicate structures such as the spinal cord. Surgeons must cut the PLL without cutting the underlying dura (lining over the spinal cord).

A number of methods have been described to cut the PLL. First, surgeons can cut the PLL directly with a knife. Second, surgeons can place a blunt object, such as a right angled probe between the dura and the PLL. After carefully protecting the dura, surgeons can cut the ligament overlying the blunt probe. Third, surgeons can use the blunt probe to tear the ligament.

### SUMMARY OF THE INVENTION

This invention resides in surgical instruments particularly suited to severing ligaments, fibrous tissues, and spinal structures. A preferred embodiment comprises a handle connected to a shaft terminating in a distal end including a blunt tip for separating tissues to be protected from tissue to be cut, and a sharp cutting blade spaced apart from the blunt tip for severing the tissue to be cut.

In alternative embodiments, the distal end may include two blunt tips, one on either side of the sharp cutting blade. For example, the shaft may terminate in a Y-shaped end including two blunt tips, with the sharp cutting blade located between the tips.

In other embodiments, the distal includes an angled portion, and the cutting blade adjoins the angled portion. For instance, the distal end may be bent, creating a longer first section coupled to the handle and a shorter portion at an angle to the first section, and the cutting blade may be located only on the first section or the second section. The angle may be less than, greater than, or substantially equal to 90 degrees.

In further embodiments, the sharp cutting blade may be retractable or covered by a moveable sheath. In all embodiments, the shaft is curved to facilitate easier viewing of the distal end. Novel retractors, other instruments, and endplates for artificial disc replacements are also disclosed.

### BRIEF DESCRIPTION OF THE DRAWINGS

FIG. 1A is a lateral view of a preferred instrument according to the present invention;

FIG. 1B is an axial cross-section of the embodiment of the invention shown in FIG. 1A;

FIG. 2 is a view of the lateral aspect of an alternative embodiment of the present invention;

FIG. 3 is a view of the lateral aspect of a further alternative embodiment of the present invention;

FIG. 4A is a view of the anterior aspect of the spine and the embodiment of the invention shown in FIG. 1A;

FIG. 4B is another view of the anterior aspect of the spine and the embodiment of the knife drawn in FIG. 1A;

FIG. 5A is a lateral view of a different alternative embodiment of the invention;

FIG. 5B is a lateral view of yet a further alternative embodiment of the invention;

FIG. 6A is a lateral view of yet a different alternative embodiment of the invention;

FIG. 6B is a lateral view of yet a different alternative embodiment of the invention;

FIG. 7A is a lateral view of a further alternative embodiment of the invention;

FIG. 7B is an end view of the instrument drawn in FIG. 7A;

FIG. 8 is a lateral view of yet a different alternative embodiment of an instrument according to the invention;

FIG. 9A is a lateral view of an alternative instrument according to the invention;

FIG. 9B is a lateral view of the instrument of FIG. 9A;

FIG. 10A is a lateral view of an alternative embodiment of the invention;

FIG. 10B is a lateral view of the embodiment of FIG. 10A;

FIG. 10C is a lateral view of yet a further alternative embodiment of the invention;

FIG. 11A is a lateral view of an alternative embodiment;

FIG. 11B is a view of the top of the sheath component drawn in FIG. 11A;

FIG. 11C is a lateral view of the embodiment of the invention drawn in FIG. 11A;

FIG. 11D is a view of the top of the cutting end of the instrument drawn in FIG. 11D;

FIG. 11E is a partial longitudinal cross section of the embodiment of the instrument drawn in FIG. 11C;

FIG. 12A is a lateral view of a different alternative embodiment of the invention;

FIG. 12B is a lateral view of the embodiment of the instrument drawn in FIG. 12A;

FIG. 12C is a partial longitudinal cross section of the embodiment of the invention drawn in FIG. 12A;

FIG. 13A is a lateral view of an alternative embodiment of the present invention;

FIG. 13B is a lateral view of the embodiment of FIG. 13B;

FIG. 13C is a view of the top of the tip of the instrument drawn in FIG. 13A;

FIG. 13D is a view of the top of the tip of the instrument drawn in FIG. 13B;

FIG. 13E is a view of the bottom of the tip of the instrument drawn in FIG. 13A;

FIG. 13F is a view of the bottom of the tip of the instrument drawn in FIG. 13B;

FIG. 13G is a lateral view of an alternative embodiment of the tip of the instrument drawn in FIG. 13A;

FIG. 13H is a lateral view of an alternative embodiment of the tip of the instrument drawn in FIG. 13B;

FIG. 13I is a lateral view of the spine and the embodiment of the instrument drawn in FIG. 13B;

FIG. 14A is an axial cross section of an intervertebral disc, the thecal sac or the dura over the spinal cord, and the nerves;

FIG. 14B is an axial cross-section of the disc and novel retractors;

FIG. 14C is an axial view of the disc, novel retractors, and an alternative embodiment of the invention;

FIG. 14D is an axial cross section of the disc and an alternative shape of the instrument drawn in FIG. 14C;

FIG. 15A is an axial cross section of an intervertebral disc and an alternative embodiment of the invention;

FIG. 15B is an axial cross section of the disc and an alternative embodiment of the invention drawn in FIG. 15A;

FIG. 15C is an axial cross section of the disc and an alternative embodiment of the invention;

FIG. 16A is a view of the top of the tip of an alternative embodiment of the invention;

FIG. 16B is a view of the top of a different tip;

FIG. 16C is a view of yet a further tip construction;

FIG. 16D is a lateral view of a portion of the handle of the device drawn in FIG. 16A;

FIG. 17A is a view of the top of an alternative embodiment of the present invention;

FIG. 17B is a view of the bottom of the instrument drawn in FIG. 17B;

FIG. 17C is a cross section of the cutting portion of the instrument drawn in FIG. 17B;

FIG. 17D is a coronal cross section of the spine and the embodiment of the invention drawn in FIG. 17C;

FIG. 17E is an axial cross section of the disc and the embodiment of the invention drawn in FIG. 17A;

FIG. 17F is view of the end of the instrument drawn in FIG. 17A;

FIG. 17G is a view of the end of an alternative embodiment of the instrument drawn in FIG. 17F;

FIG. 17H is a view of the end of an alternative embodiment of the instrument drawn in FIG. 17G;

FIG. 17I is a view of the end of an alternative embodiment of the instrument drawn in FIG. 17H;

FIG. 18A is an axial cross section of a disc and prior art ADR endplate;

FIG. 18B is an axial cross section of a disc and a novel ADR EP;

FIG. 19A is an axial view of an intervertebral disc;

FIG. 19B is also an axial view of an intervertebral disc;

FIG. 20A is a superior view of an ADR EP with a novel shape;

FIG. 20B is a superior view of an alternative embodiment of the ADR EP drawn in FIG. 20A;

FIG. 21A is a lateral view of the spine;

FIG. 21B is a lateral view of the spine and the embodiment of the invention drawn in FIG. 21A;

FIG. 21C is a superior view of an instrument according to the invention that may be used to create the horizontal cuts drawn in FIG. 21A;

FIG. 21D is an end view of the cutting surfaces of the embodiment of the invention drawn in FIG. 21C;

FIG. 21E is a superior view of an alternative embodiment of the invention drawn in FIG. 21C; and

FIG. 21F is an end view of the cutting surface of the embodiment of the invention drawn in FIG. 21E.

### DETAILED DESCRIPTION OF THE INVENTION

This invention improves on the prior art by providing a posterior longitudinal ligament (PLL) knife with a cutting component incorporated into a blunt probe. Generally speaking, the blunt tip of the knife is used to develop a plane between the dura and the PLL, and the trailing edge of the knife is used to cut the PLL. In the preferred embodiment, the blunt probe-like portion of the knife is perpendicular to the handle of the knife, though other configurations are disclosed. The shape of the knife facilitates work through a small incision (approximately 1 inch) overlying the entrance of a deep hole (i.e., 2 or more inches).

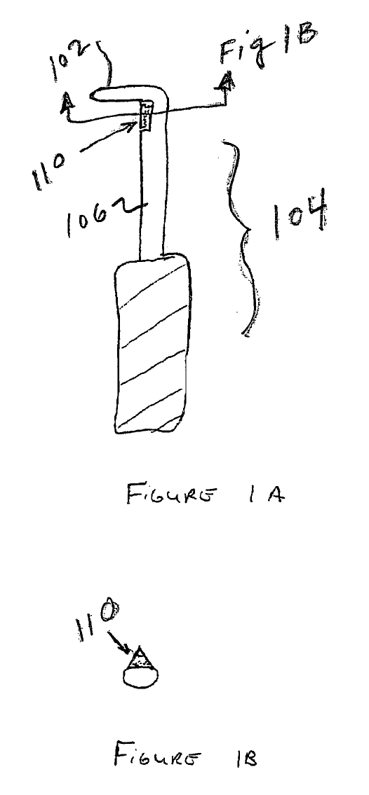

FIG. 1A is a view of the lateral aspect of a preferred embodiment according to the invention. A blunt, probe-like member 102 extends generally perpendicular to the handle 104 of the PLL knife. A cutting component 110 lies along the edge of the shaft 106 of the instrument. The blunt tip is used to develop a plane between the structure to be cut and the underlying tissues. The sharp edge of the knife cuts the tissues that lie directly above the blunt tip as lateral pressure is applied to the knife. FIG. 1B is an axial cross section of the embodiment of the invention drawn in FIG. 1A.

FIG. 2 is a view of the lateral aspect of an alternative embodiment of the invention, wherein the cutting component 202 extends along the probe-like section 204. The instrument may include a “bayonet” shaped section 206 to facilitate a view of the tip of the instrument. Tissues are cut as the knife is pulled in the direction of the handle 208.

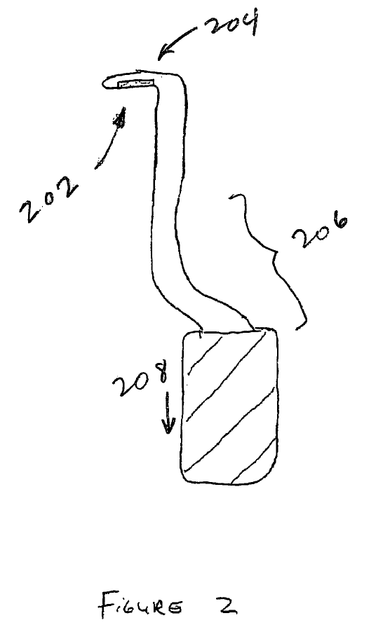

FIG. 3 is a view of the lateral aspect of an alternative embodiment of the invention, wherein the angle 300 between the probe-like component and the handle of the instrument is somewhat greater than 90 degrees. The angle could also be somewhat less than 90 degrees.

FIG. 4A is a view of the anterior aspect of the spine and the embodiment of the invention drawn in FIG. 1A illustrating the use of the blunt tip of the knife to develop a plane between the PLL (vertical lines) and the underlying dura and spinal cord. FIG. 4B is another view of the anterior aspect of the spine and the embodiment of the knife drawn in FIG. 1A, wherein a portion of the PLL has been cut by the knife.

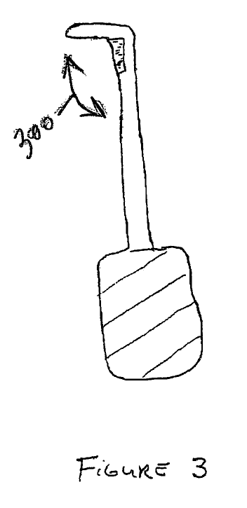

FIG. 5A is a lateral view of an alternative embodiment of the invention in the form of a knife having two blunt projections 502, 504 from its leading edge. The sharp knife component 510 lies between the two blunt projections. The longer blunt projection 504 is used to dissect between the PLL and the dura. The blunt portion of the longer projection is preferably at least 0.2 mm long. The shorter blunt projection 502 lies above the PLL. The shorter projection keeps the knife from plunging into the spinal canal. The handle of the instrument may be angled or bayoneted to improve the surgeon's view of the tip. The instrument is designed for incising the PLL for an anterior approach to the spine, and may have cervical and lumbar embodiments. The cervical embodiment is much thinner. The diameter of the blunt projections on the cervical embodiment is less than 2 mm. The diameter of the blunt projections on the lumbar embodiment are 2 mm or greater. The tip of the longer blunt projection may have a small point to ease the probe like tip between the tissues. The vertebrae may be distracted to place the PLL under tension. The PLL is easier to cut when the fibers are placed under tension.

FIG. 5B is a lateral view of an instrument shaped for use in a posterior, lateral, or posterior lateral approach to the spine. The instrument is also an alternative embodiment of the instruments described in my co-pending U.S. patent application Ser. No. 10/864,160 entitled “Treating Disc Herniation And Other Conditions With Leukocytes,” the entire content of which is incorporated herein by reference.

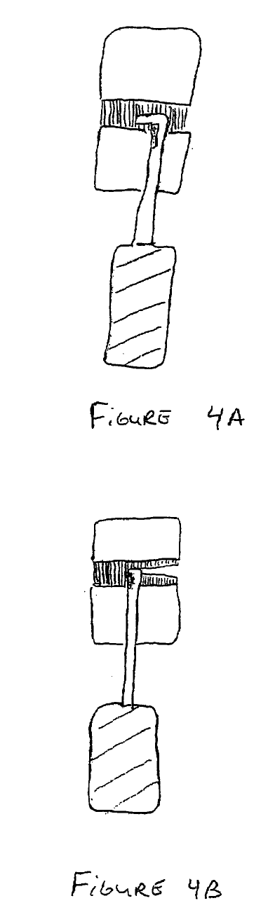

FIG. 6A is a lateral view of an alternative embodiment of the invention having blunt projections 602, 604 which are angled differently than the projections drawn in FIG. 5B. FIG. 6B is a lateral view of an alternative embodiment of the invention having an upper blunt projection 612 which is angled relative to the shaft of the instrument. FIG. 7A is a lateral view of an alternative embodiment, wherein the cutting portion 710 of the instrument lies below a blunt probe 720. FIG. 7B is an end view of the instrument drawn in FIG. 7A.

FIG. 8 is a lateral view of an alternative embodiment in the form of a hook-like instrument wherein the cutting edge 802 is below the blunt tip 804 and directed towards the handle of the instrument.

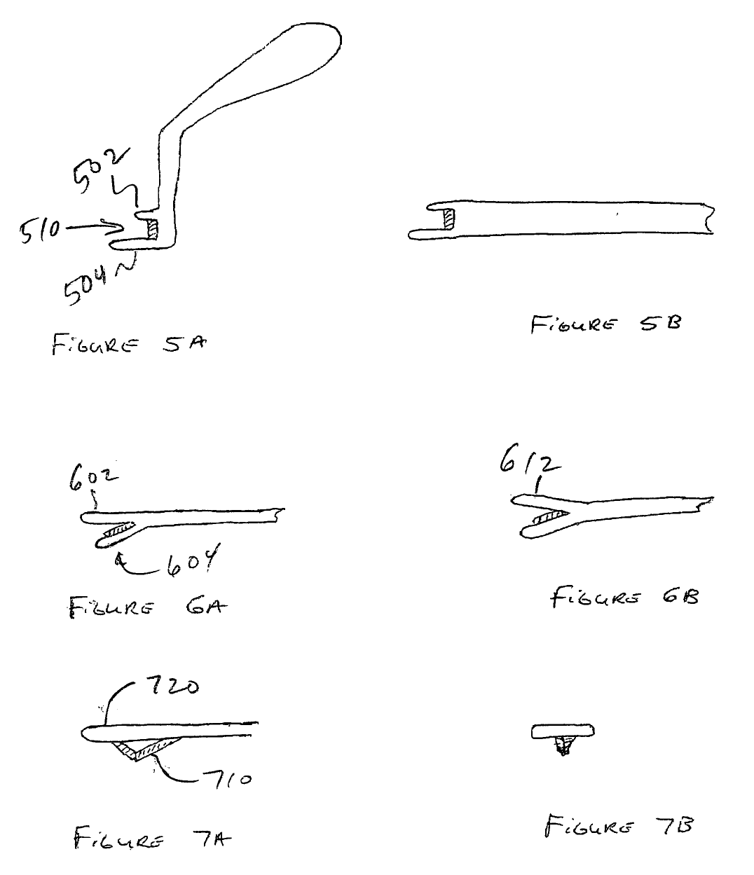

FIG. 9A is a lateral view of an alternative embodiment of an instrument with a retractable cutting component. The instrument was drawn with the cutting component retracted into the handle component. FIG. 9B is a lateral view of the embodiment of the invention drawn in FIG. 9A with the cutting component 902 in the extended or exposed position.

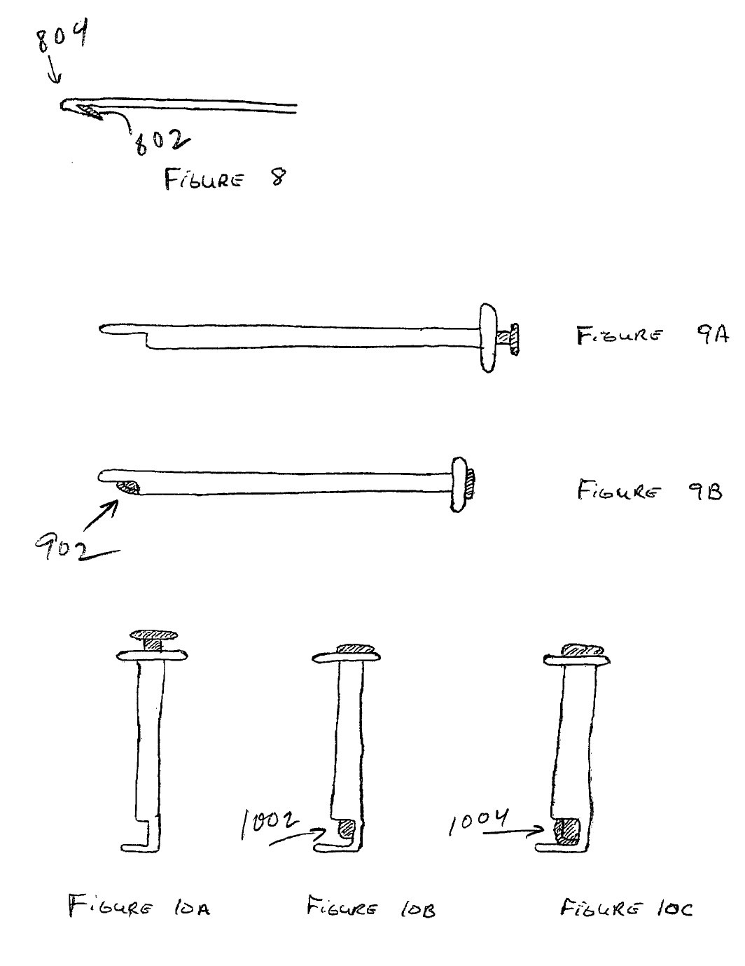

FIG. 10A is a lateral view of an alternative embodiment also having a retractable cutting component. The instrument was drawn with the cutting component retracted into the handle of the device. FIG. 10B is a lateral view of the embodiment of the invention drawn in FIG. 10A. The instrument was drawn with the cutting component 1002 exposed. FIG. 10C is a lateral view of an alternative embodiment of the invention drawn in FIG. 10B wherein a cutting component 1004 extends to the blunt probe in the exposed position.

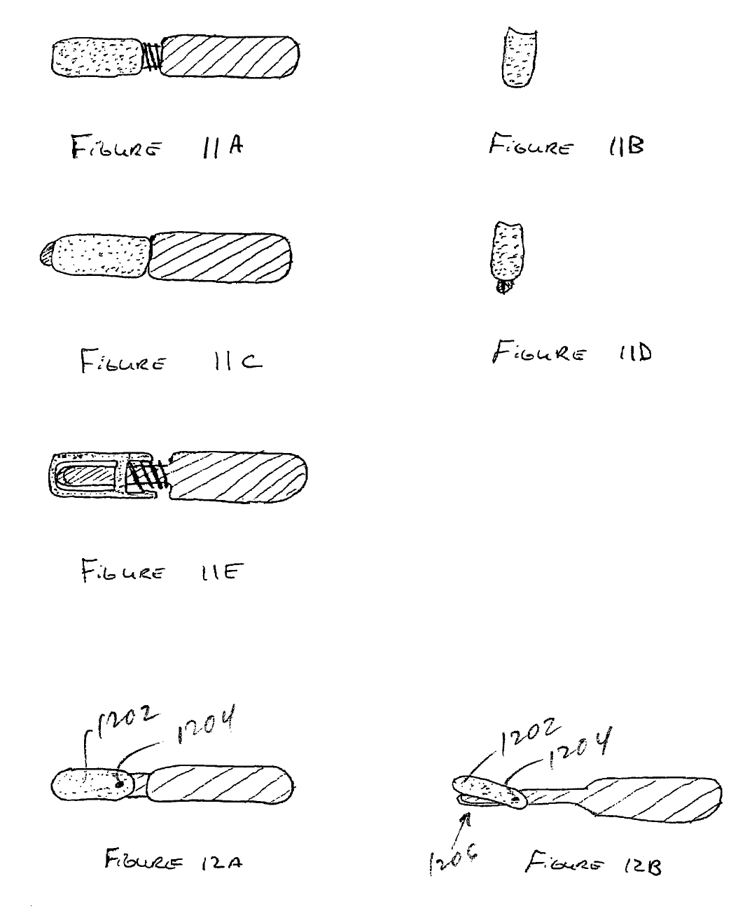

FIG. 11A is a lateral view of an alternative embodiment of an instrument with its blunt, spring loaded sheath component covering the cutting edge of the instrument. FIG. 11B is a view of the top of the sheath component drawn in FIG. 11A. FIG. 11C is a lateral view of the embodiment of the invention drawn in FIG. 11A. The instrument is drawn with the sheath retracted, thus exposing the cutting tip of the instrument. The sheath retracts when pressure is applied to the tip of the instrument. The sheath retracts when the instrument is forced against the PLL, Annulus Fibrosus (AF) or other spinal ligament. The sheath returns to cover the cutting portion of the instrument as the instrument cuts through the ligament. The sheath component impinges against the handle component to limit how much of the cutting tip is exposed. The instrument may be used to cut spinal ligaments while protecting the underlying nerves. FIG. 11D is a view of the top of the cutting end of the instrument drawn in FIG. 11D. FIG. 11E is a partial longitudinal cross section of the embodiment of the instrument drawn in FIG. 11C.

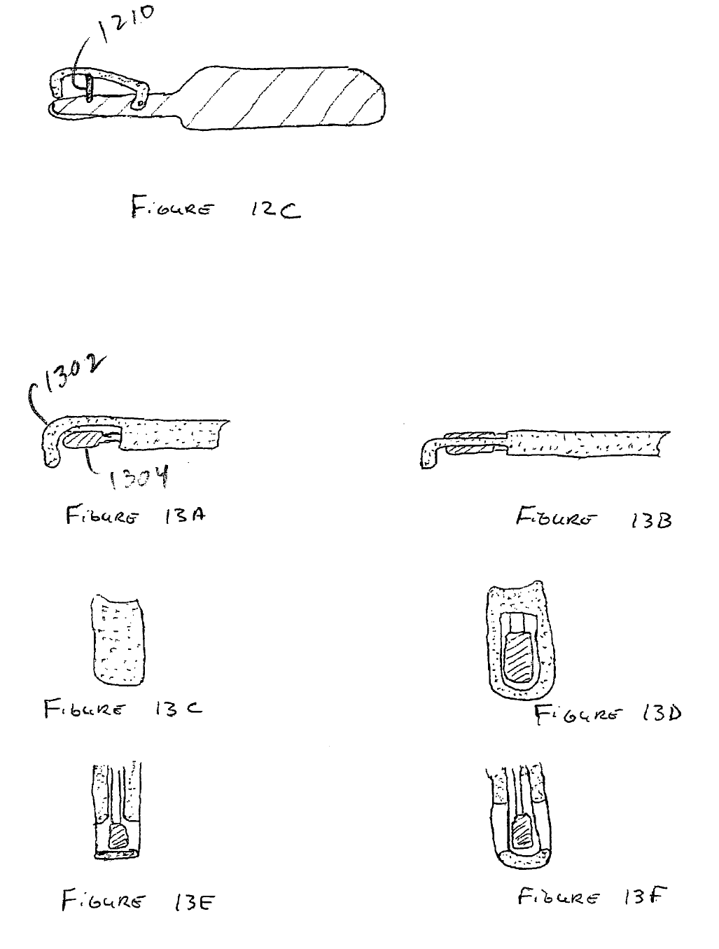

FIG. 12A is a lateral view of an alternative embodiment of an instrument wherein a sheath component 1202 is attached to an axle 1204 that courses through the cutting component 1206 of the instrument. FIG. 12B is a lateral view of the embodiment of the instrument drawn in FIG. 12A. The sheath component is rotated to expose the cutting component of the instrument. The cutting component of the instrument is exposed by applying pressure to the side of the sheath. FIG. 12C is a partial longitudinal cross section of the embodiment of the invention drawn in FIG. 12A. An elastic cord 1210 extends from the sheath component to the cutting component.

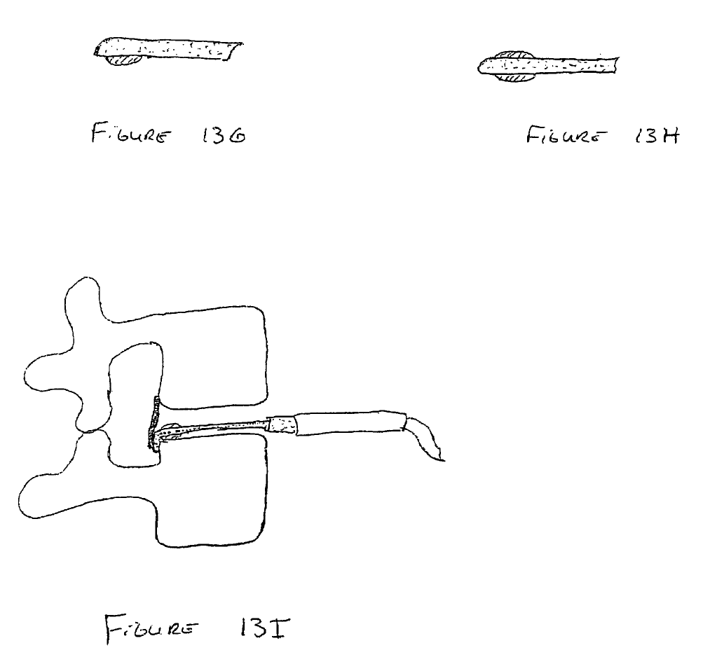

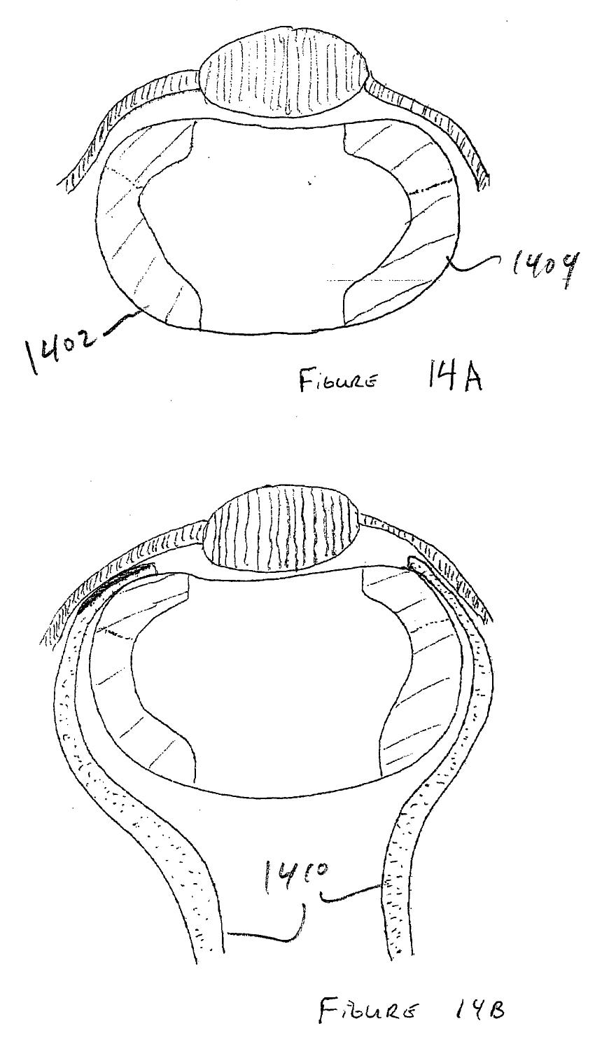

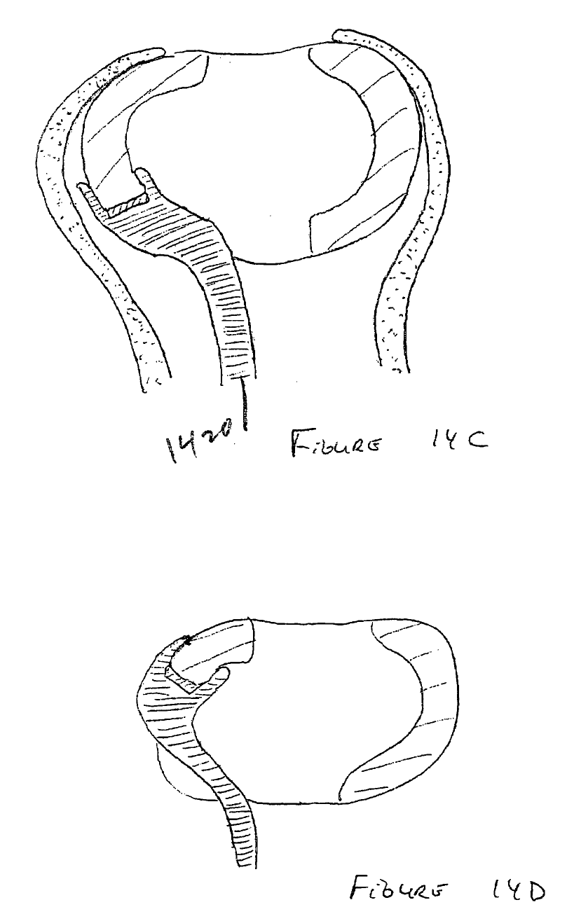

FIG. 13A is a lateral view of an alternative embodiment of the invention. A blunt probe component 1302 extends beyond the tip of a power bur 1304. The probe protects the nerves from the spinning bur. The probe may be used to dissect between the PLL and the vertebrae. The bur may be used to remove a portion of the vertebral body. FIG. 13B is a lateral view of an alternative embodiment wherein the probe component of the device is smaller to improve the surgeon's view of the tip of the instrument. FIG. 13C is a view of the top of the tip of the instrument drawn in FIG. 13A. FIG. 13D is a view of the top of the tip of the instrument drawn in FIG. 13B.

FIG. 13E is a view of the bottom of the tip of the instrument drawn in FIG. 13A. FIG. 13F is a view of the bottom of the tip of the instrument drawn in FIG. 13B. FIG. 13G is a lateral view of an alternative embodiment of the tip of the instrument drawn in FIG. 13A. The probe extends around the sides of the bur. The bur may have a flat side to aid insertion of the bur into the sheath of instrument. FIG. 13H is a lateral view of an alternative embodiment of the tip of the instrument drawn in FIG. 13B. FIG. 13I is a lateral view of the spine and the embodiment of the instrument drawn in FIG. 13B. The probe component of the instrument can be seen between the PLL, or the dura, and the body of the vertebra.

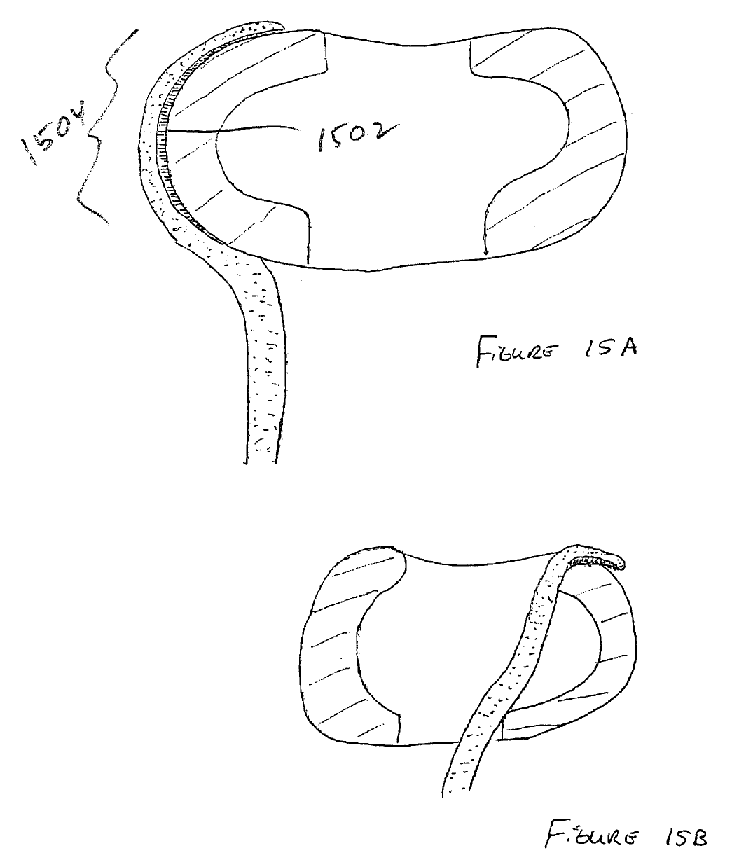

FIG. 14A is an axial cross section of an intervertebral disc, the thecal sac or the dura over the spinal cord, and the nerves. A portion of the AF has been removed, and areas 1402, 1404 represent the remaining AF. The amount of resected AF is similar to prior-art methods of inserting prior art artificial disc replacements (ADRs). The anterior and the posterior portions of the AF are removed during insertion of prior art ADRs. The dotted line through the AF represents the border between the “safe” and “nerve” areas of the AF. The portion of the AF that lies anterior to the dotted lines may be removed or incised with little fear of injuring the nerves. The spinal nerves are at risk of injury when excising or incising the AF posterior to the dotted lines. The novel method removes (excises), incises, or releases more AF than removed in prior art methods of inserting ADRs. Removing or releasing additional AF improves the mobility of the spine, decreases the risk of pain from injured AF, and increases the surface of area of exposed vertebral endplate (VEP). Prior art methods of soft tissue release often involve tearing the AF and the ligaments by impacting distractors into the disc space. The novel “soft tissue” release is less traumatic to the vertebrae and the nerves.

FIG. 14B is an axial cross section of the disc and retractors 1410 according to the invention. The retractors have been placed between the lateral and posterior lateral portions of the disc and the nerves. The tip of the retractors may be electrified. As described in my co-pending U.S. patent application Ser. No. 10/842,192, the entire content of which is incorporated herein by reference. For example, the nerve conduction velocity (NCV) and the amplitude of the recorded response from the extremities could be monitored during the procedure to detect early signs of nerve injury. Nerve injury may occur from excessive spinal distraction or from pressure on a nerve. Excessive distraction may occur from preparation of the disc space for ADR insertion or from a large ADR. Free run emgs may also be recorded from the extremities to avoid nerve injury. The dark lines on the tip of the retractor drawn on the left side of the drawing represent the portion the retractor that emits electrical impulses. The remaining portion of the retractor could be insulated to avoid shunting. An endoscope may be used to improve visualization while placing the retractors.

FIG. 14C is an axial view of the disc, the novel retractors, and an embodiment of an instrument 1420 is used to incise the AF. The AF is much thicker and much tougher than the PLL. Thus, the instrument is typically larger and more robust than the embodiments disclosed to incise the PLL. The instrument is also preferably shaped to help direct the instrument in the course of the AF. The disc space may be distracted. The AF is easier to release when the fibers are placed under tension. FIG. 14D is an axial cross section of the disc and an alternative shape of the instrument drawn in FIG. 14C. The instrument is shaped to incise the posterior-lateral portion of the AF.

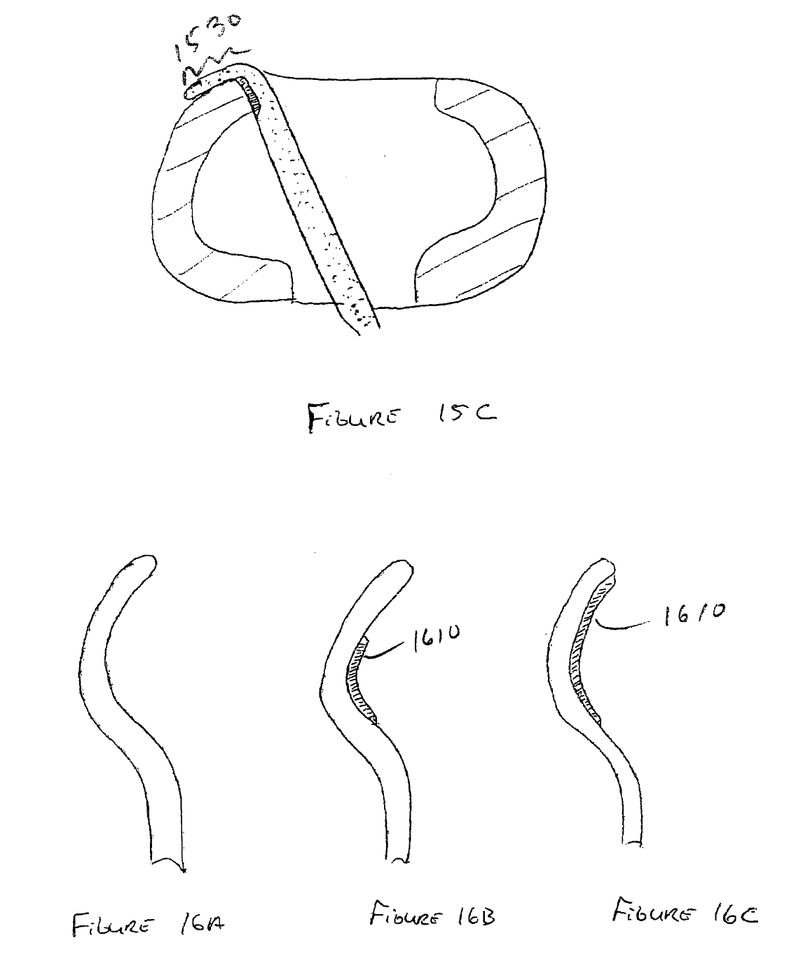

FIG. 15A is an axial cross section of the disc and a semi-circular knife is used to cut the AF. The blade is shown at 1502, and lateral portion 1504 of the knife is blunt to prevent nerve injury. FIG. 15B is an axial cross section of the disc and an alternative embodiment of the invention drawn in FIG. 15B. The knife is shaped to incise the AF from inside the disc. As mentioned previously, the knife is substantially larger than a knife used to cut the PLL, especially the thin PLL in the cervical spine. The novel method may include cutting the posterior AF and a portion of the lateral AF. At least a portion of the lateral AF may be preserved.

FIG. 15C is an axial cross section of the disc and an alternative embodiment of the invention drawn in FIG. 15B. The knife is shaped to incise the AF from inside the disc. The knife has a blunt probe-like tip 1530. This embodiment of the invention may also have two probe tips similar to those drawn in FIG. 5A.

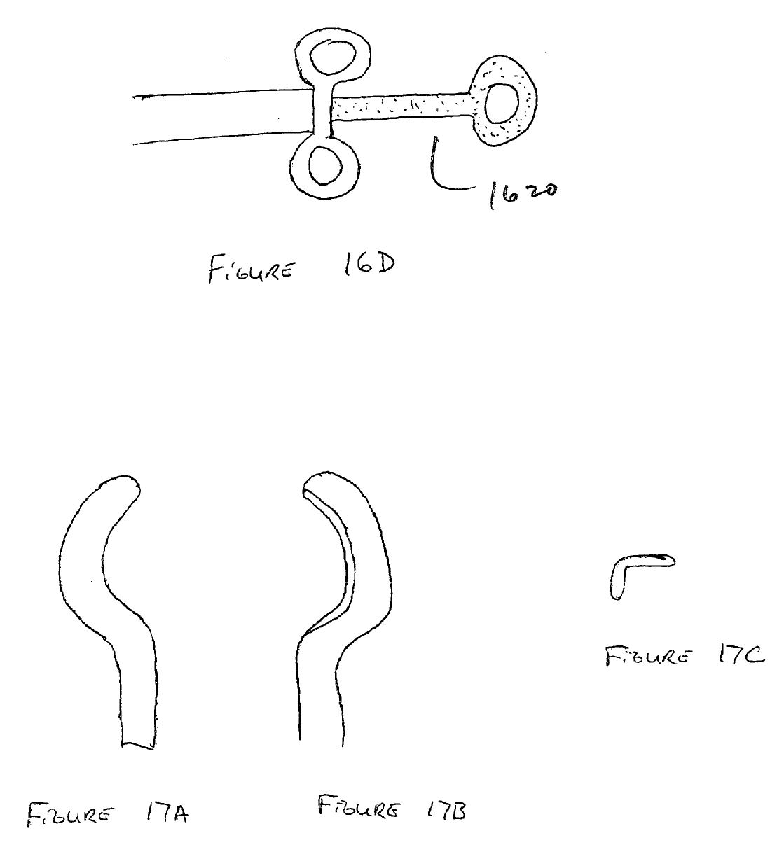

FIG. 16A is a view of the top of the tip of a knife used to incise the AF. The cutting portion of the knife is retracted into the handle of the knife. FIG. 16B is a view of the top of the embodiment of the invention drawn in FIG. 16A. The cutting component 1610 is drawn in its partially exposed position. FIG. 16C is a view of the top of the embodiment of the invention drawn in FIG. 16B. The cutting component 1610 is drawn in its fully extended position. Alternatively, this embodiment of the invention could utilize a retractable guard than exposes increasing portions of the semi-circular cutting blade. FIG. 16D is a lateral view of a portion of the handle of the device drawn in FIG. 16A. The retractable blade (or retractable sheath) is moved by advancing or retracting the plunger-like component 1620.

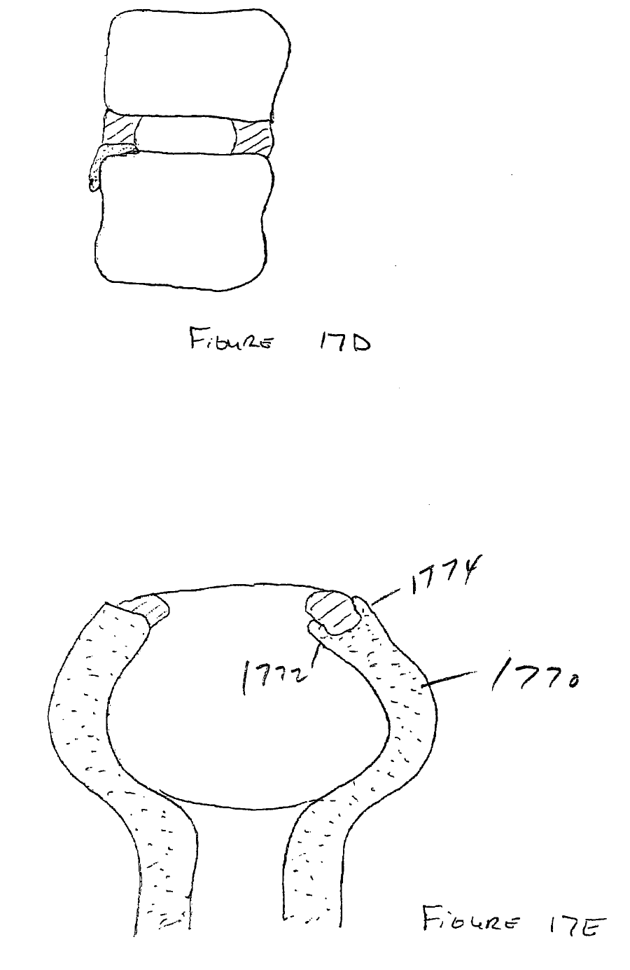

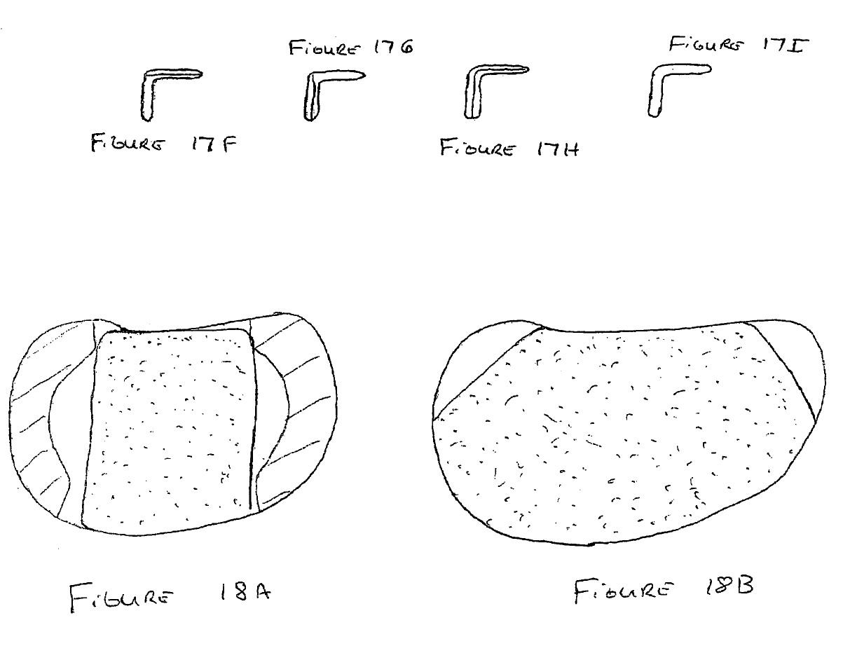

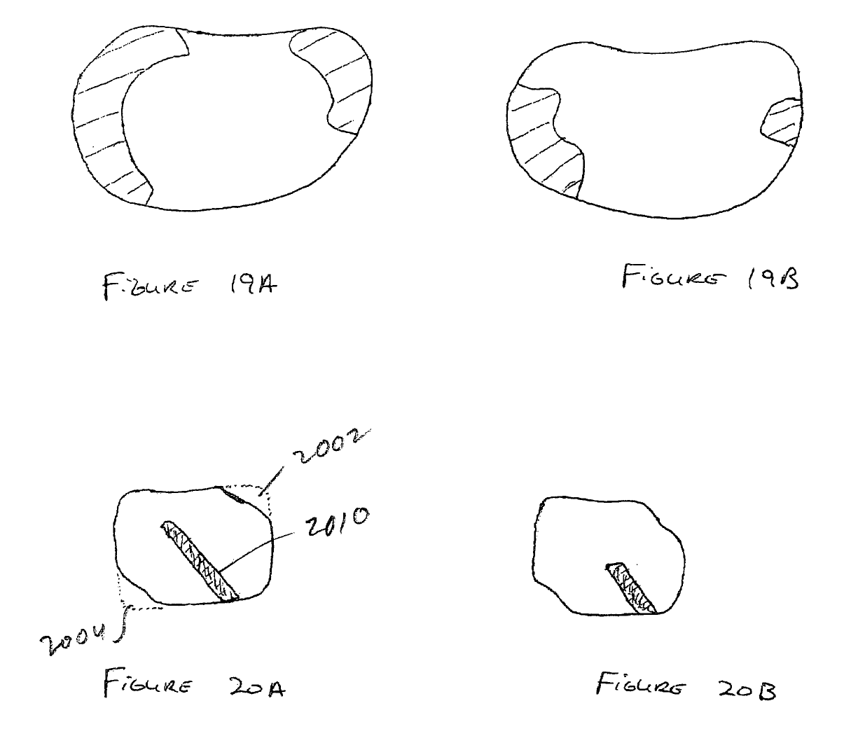

FIG. 17A is a view of the top of a novel semi-circular shaped cutting instrument. FIG. 17B is a view of the bottom of the instrument drawn in FIG. 17B. FIG. 17C is a cross section of the cutting portion of the instrument drawn in FIG. 17B. FIG. 17D is a coronal cross section of the spine and the embodiment of the invention drawn in FIG. 17C. The instrument is guided between the AF superior and lateral edges of the VEP. The instrument releases the AF from the VEP. The instrument preferably leaves a sleeve of AF or ligament tissue that surrounds the lateral portion of the vertebra. FIG. 17E is an axial cross section of the disc and the embodiment of the invention drawn in FIG. 17A. The instruments have been directed between the AF and the VEP. The instrument 1770 has blunt probes 1772, 1774 that project beyond the cutting surface of the instrument.

FIG. 17F is view of the end of the instrument drawn in FIG. 17A. The edge of the horizontal portion of the instrument is beveled along its center. FIG. 17G is a view of the end of an alternative embodiment of the instrument drawn in FIG. 17F. The vertical portion of the instrument is beveled along its center. FIG. 17H is a view of the end of an alternative embodiment of the instrument drawn in FIG. 17G. The vertical and the horizontal portions of the instrument are beveled along their centers. FIG. 17I is a view of the end of an alternative embodiment of the instrument drawn in FIG. 17H. The vertical portion, the horizontal portion, or both portions of the instrument are beveled along the edges of the components rather than along the components centers.

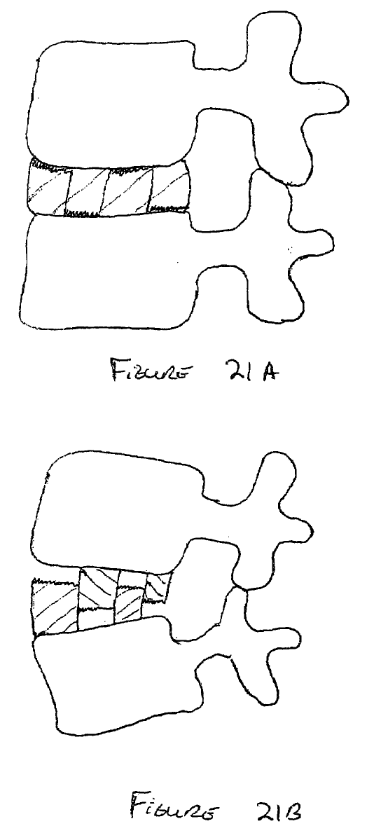

FIG. 18A is an axial cross section of a disc and prior art ADR endplate (ADR EP). Prior art ADR EPs are shaped to fit the exposed area of the VEP after using prior art methods. The ADR EPs generally have straight posterior edges. Prior art ADR EPs do not cover the VEP than lies beneath or above the retained AF. FIG. 18B is an axial cross section of a disc and a novel ADR EP. The ADR EP covers more VEP than prior art ADR EPs. Excision of more AF with the novel methods taught in this application enable the use of ADRs with larger EPs. ADRs that cover a larger portion of the VEPs gain the advantage of the increase support by the VEPs. The increased surface area facilitates bone ingrowth into the ADR EP, decreases the risk of fracturing the VEPs, and provides additional friction between the ADR EP and the VEP. The posterior lateral corners of the novel ADR EP are notched to minimize the risk of nerve root impingement. The posterior edge of the novel ADR EP is concave to maximize coverage of the VEP.

FIG. 19A is an axial view of an intervertebral disc, wherein a section of the lateral portion of the AF has been removed to facilitate insertion of ADRs with oblique keels, as described in my co-pending U.S. patent application Ser. No. 10/860,920, entitled “Methods and Apparatus for Total Disc Replacements with Oblique Keels,” the entire content of which is incorporated herein by reference.

FIG. 19B is an axial view of an intervertebral disc wherein posterior and posterior-lateral portions of the AF have been removed to facilitate spinal flexion. A section of the lateral portion of the AF has been removed to facilitate insertion of ADRs with oblique keels. A section of the lateral portion of the AF has been preserved.

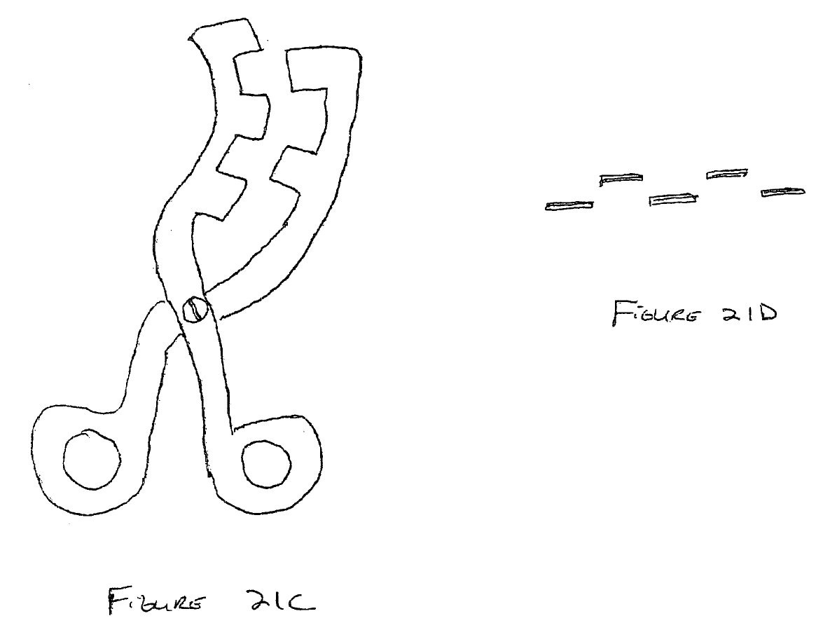

FIG. 20A is a superior view of an ADR EP with a novel shape. The dotted lines represents the shape of prior-art ADR EPs. The novel shape, which removes two corners 2002, 2004 of prior-art ADRs, facilitates insertion of ADRs with oblique keels. The novel shape reduces the amount of AF that must be removed to insert the ADR. A novel keel 2010 is preferentially located closer to the anterior portion of the ADR than the posterior portion of the ADR. The anterior location of the keel facilitates ADR insertion. FIG. 20B is a superior view of an alternative embodiment of the ADR EP drawn in FIG. 20A. The keel is limited to the anterior half of the ADR EP.

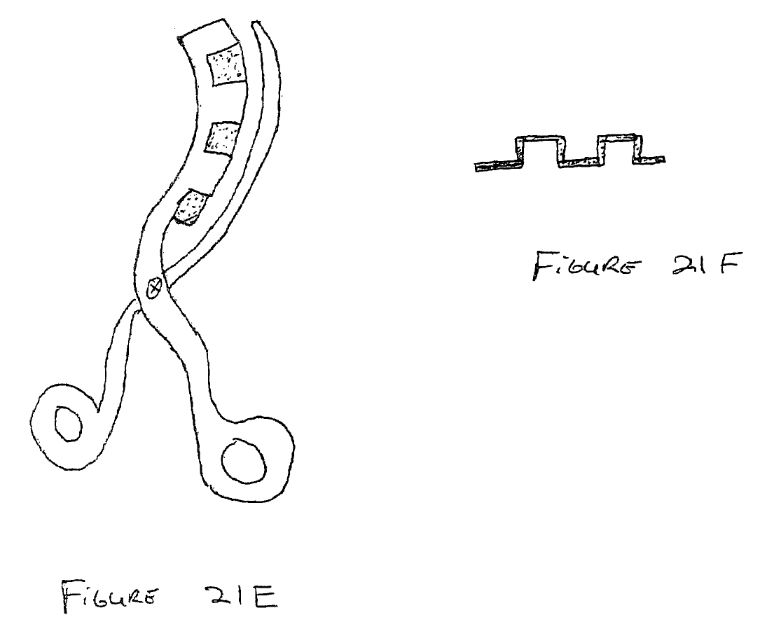

FIG. 21A is a lateral view of the spine, wherein the AF is represented by the area of the drawing with diagonal lines. Portions of the AF have been released from the superior and inferior vertebrae. The released sections alternate between the superior and inferior portions of the AF. FIG. 21B is a lateral view of the spine and the embodiment of the invention drawn in FIG. 21A. The disc space has been distracted. The released portions of the AF separate from the superior and the inferior vertebrae in an alternating fashion. The released portions of the AF slide along tears between the portions of the AF. The vertical tears occur during distraction of the disc space. The disc space is distracted after making the horizontal cuts. Alternatively, the vertical components may be surgically created.

This embodiment of the invention may be used on ligaments, the AF, and other tissues that surround any portion of the spine. The ligaments and the AF may be cut in other ways that allow the soft tissues to be “lengthened”, for example, the soft tissues may be lengthen via the plastic surgery techniques known as Z-plasty and V-Y advancement. The soft tissues about the spine may also be lengthened with oblique cuts through the tissue. This embodiment anticipates any mechanism that cuts or tears the soft tissues about that spine, thus allowing the vertebrae to separate, and yet maintain at least partial overlap or connection of the lengthened ligament or AF. FIG. 21C is a superior view of a novel instrument that may be used to create the horizontal cuts drawn in FIG. 21A. FIG. 21D is an end view of the cutting surfaces of the embodiment of the invention drawn in FIG. 21C. The instrument may be adjusted to vary the vertical distance between the superior and the inferior cutting blades. The instrument would also be supplied to surgeons in various sizes.

FIG. 21E is a superior view of an alternative embodiment of the invention drawn in FIG. 21C. The cutting portion of the instrument is limited to one side of the device. The device is clamped around the portion of the AF or other spinal ligament that is to be released. Various shapes of the instrument may be manufactured. FIG. 21F is an end view of the cutting surface of the embodiment of the invention drawn in FIG. 21E. The instrument makes vertical and horizontal cuts.

1. A surgical instrument particularly suited to severing ligaments, fibrous tissues, and spinal structures, comprising:
a handle connected to a shaft terminating in a distal end including a blunt tip for separating tissues to be protected from tissue to be cut; and
a sharp cutting blade spaced apart from the blunt tip for severing the tissue to be cut.

2. The surgical instrument of claim 1, wherein the distal end includes two blunt tips, one on either side of the sharp cutting blade.

3. The surgical instrument of claim 1, wherein:
the shaft terminates in a Y-shaped end including two blunt tips; and
the sharp cutting blade is located between the tips.

4. The surgical instrument of claim 1, wherein:
the distal includes an angled portion; and
the cutting blade adjoins the angled portion.

5. The surgical instrument of claim 1, wherein:
the distal end is bent creating a longer first section coupled to the handle and a shorter portion at an angle to the first section; and
the cutting blade is only on the first section.

6. The surgical instrument of claim 1, wherein:
the distal end is bent creating a longer first section coupled to the handle and a shorter portion at an angle to the first section; and
the cutting blade is only on the second section.

7. The surgical instrument of claim 1, wherein the angle is less than, greater than, or substantially equal to 90 degrees.

8. The surgical instrument of claim 1, wherein the sharp cutting blade is retractable.

9. The surgical instrument of claim 1, wherein the sharp cutting blade is covered by a moveable sheath.

10. The surgical instrument of claim 1, wherein the shaft is curved to facilitate easier viewing of the distal end.
